(19) United States
(12) Patent Application Publication
(10) Pub. No.: US 2014/0236188 A1
(43) Pub. Date: Aug. 21, 2014

(54) INTRODUCER SHEATH WITH KNOT PUSHER AND SUTURE TRIMMER

(71) Applicant: Abbott Cardiovascular Systems, Inc., Santa Clara, CA (US)

(72) Inventors: Douglas H. Mehl, Redwood City, CA (US); Aaron M. Fortson, Fremont, CA (US); Richard J. Rotzman, New York, NY (US); Dana G. Vaughn, Boston, MA (US)

(73) Assignee: Abbott Cardiovascular Systems, Inc., Santa Clara, CA (US)

(21) Appl. No.: 13/769,015

(22) Filed: Feb. 15, 2013

(51) Int. Cl. A61B 17/04

(52) U.S. Cl. CPC A61B 17/0469
USPC 606/144

(57) ABSTRACT

A device configured as a knot pusher and/or a suture trimmer is provided. The device can deliver and secure a suture knot. The apparatus can also trim the suture after the suture knot is secured.

### BACKGROUND OF THE INVENTION

In many medical procedures, there is often a need to introduce a medical device inside of a patient's body. For example, an angioplasty includes the placement of a stent inside of a vessel. As a result, there is a need to access the lumen of the vessel with various medical devices that enable the stent to be appropriately placed. During the angioplasty, an opening in the vessel (e.g., an arteriotomy) is formed. Access to the vessel's lumen is achieved through the opening. After the procedure is completed, it then becomes necessary to close the opening formed in the vessel.

More generally, the closure of openings (or other incisions or wounds) in vessels or tissue, can be closed using several different techniques. Staples, tissue glues, and clips are examples of devices or materials used to close openings in tissue.

In addition to these devices, suture may also be used to close openings. In fact, many surgeons and physicians prefer the use of suture because suture tightly and reliably closes openings in vessels or tissue. Plus, the surgeon or physician placing the suture is likely to know immediately whether the suture will fail or hold. Advantageously, if the suture fails, the surgeon or physician can immediately redo the suture.

In contrast, other techniques may fail on occasion. Unfortunately, the failure of these devices may not be known until some time after the procedure has been performed. In this case, the surgeon or physician may no longer be available to remedy the failure of the device. As a result, the use of suture is preferred by some surgeons and physicians.

At the same time, suture is not without problems. When closing an opening that is below the patient's skin, the puncture site may need to be enlarged in order to allow the suture to be tightly secured and trimmed. Enlarging the puncture site is not desirable. Trimming the suture above the patient's skin is also undesirable.

U.S. Pat. No. 6,746,457 discloses a snared suture trimmer that can be used to trim a suture. This trimmer, however, includes multiple parts (e.g., shafts and fittings) that work together to trim the suture. Because there are several parts, the operating mechanism of the device is more complex. In addition, the costs of manufacturing are increased due in part to the number of parts and the need to assemble the various parts.

### BRIEF SUMMARY OF THE INVENTION

Embodiments relate to systems, devices, and/or methods for delivering and securing suture. In one example, a device for delivering and securing a suture is provided. The device includes a shaft assembly extending between a distal end and a proximal end of the device. The shaft assembly includes a sheath and a shaft slidably disposed inside the sheath. In one example, only the shaft is disposed inside the sheath. The shaft occupies the entire lumen of the sheath with a close tolerance between the outer perimeter of the shaft and the inner perimeter of the sheath in one example. The distal end of the device is configured to push a knot formed in the suture and the shaft assembly is configured to trim the suture once the knot is delivered and secured. This configuration reduces the number of parts needed for delivering, securing, and/or trimming suture and can also be sized to prevent a puncture site from being enlarged.

In another example, the device includes a distal end with a tip configured to push a knot in the suture and a proximal end. A housing is formed on the proximal end of the device and a bias mechanism is located within the housing. A shaft assembly extends between the distal end and the proximal end of the device. The shaft assembly includes a sheath that has a suture access port, which may be located adjacent the distal end of the shaft assembly. The tip and the sheath cooperate to form a suture path through the device. The shaft assembly also includes a shaft slidably disposed inside the sheath. In some embodiments, only the shaft is slidably disposed inside the sheath and the shaft, which may have a solid body, occupies an entire lumen of the sheath.

In one example, the shaft is configured to fit inside a lumen of the sheath. The inner diameter (or perimeter) of the lumen and the outer diameter (or perimeter) of the shaft are selected to have a close tolerance relationship while allowing the shaft to move axially relative to the sheath.

To operate the device, a distal end of the shaft is advanced distally. The distal end of the shaft cooperates with the suture access port (e.g., by scissor action) to trim the suture after the knot is tightened. The bias mechanism in the housing prevents the shaft from being prematurely advanced distally. The shaft passes through the housing and a proximal end of the shaft extending proximally out of the housing is configured such that a user can advance the shaft distally. The proximal end of the shaft may have a push button, a handle, or the like, that can be grasped and pushed or operated by a user to advance the shaft distally relative to the sheath.

Embodiments of the invention provide a device that can snare and trim sutures using fewer parts. Embodiments of the invention can be used to snare sutures, advance knots, and trim the sutures using simplified mechanisms with fewer parts. These differences, compared to conventional devices, reduce manufacturing costs, are easier to operate and may provide more effective trimming while reducing trauma to the access site or the puncture being closed.

These and other advantages and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

The invention will be described and explained with additional specificity and detail through the use of the accompanying drawings in which:

FIG. 1 illustrates a perspective view of a device configured as a knot pushing device and/or a suture trimming device;

FIG. 2 illustrates a cross sectional view of the device illustrated in FIG. 1;

FIG. 3 illustrates a perspective view of a distal end of the device;

FIG. 4 illustrates one example of a cross sectional view of the distal end of the device;

FIG. 5 illustrates another example of a cross sectional view of the distal end of the device;

FIGS. 6-10 illustrate an exemplary method for pushing a knot to an opening and trimming the sutures after tightening the knot.

### DETAILED DESCRIPTION OF THE PREFERRED EMBODIMENTS

Embodiments of the invention relate to systems and methods for delivering and securing suture knots. Embodiments further relate to delivering and securing suture knots to close an opening such as an arteriotomy without over-dilating a puncture site. Advantageously, the device may be configured such that the suture can be trimmed below the puncture site.

In one example, a device may include a suture snare configured to capture or snare ends of a suture in which a knot has been formed. After the suture ends are snared by the suture snare, the suture snare can then be used to load the suture in the device. Once the suture is loaded in the device, the knot formed in the suture can be pushed down to the arteriotomy and tightened. Once the knot is tightened, the device may be configured to trim the suture near the knot. In one example, the sutures are trimmed below the puncture site or at another location.

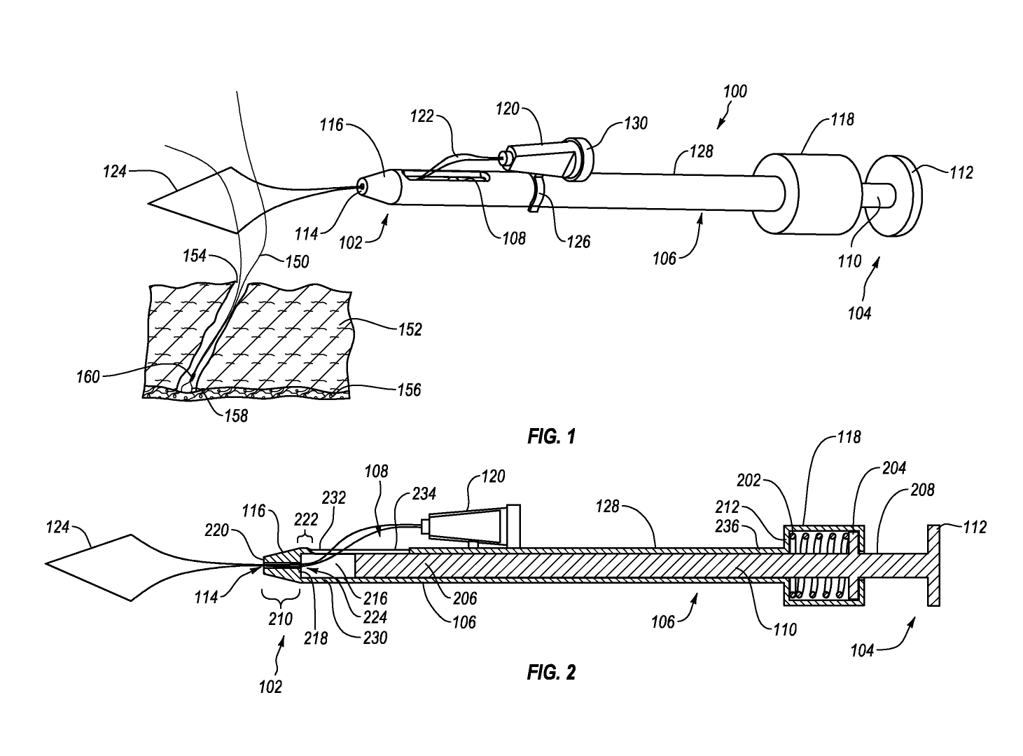

FIG. 1 illustrates a perspective view of a device 100 that is configured as a knot pusher and/or a suture trimmer. The device 100 can be used to close an arteriotomy or other opening in tissue or other material and can be used for the benefit of humans and non-humans.

When performing a procedure (e.g., an angioplasty to deliver a stent), access to a vessel 156 is required because the procedure is performed within a lumen of the vessel 156. Access to the vessel 156 is achieved through a puncture site 154 in the tissue 152.

In FIG. 1, an arteriotomy 158 has been formed in the vessel 156 (e.g., the femoral artery) for a procedure. After the procedure when the medical devices used during the procedure are withdrawn from the vessel 156 and the arteriotomy 158 is ready to be closed, suture 150 may be placed in the vessel walls near or adjacent the arteriotomy 158. A knot 160 (which may be pre-formed) is present in the suture 150.

The proximal ends of the suture 150 are then loaded in the device 100. After the suture 150 is loaded in a distal end 102 of the device 100, the device 100 can then be operated to push the knot 160 toward the arteriotomy 158. Once the knot 160 is pushed to the appropriate location and the knot 160 is tightened (e.g., by pulling on one or both proximal ends of the suture 150), the device 100 can be operated to trim the suture 150. The suture 150, in one example, is preferably trimmed below the puncture site 154 and near the arteriotomy 158.

The device 100 used to secure the knot 160 includes a distal end 102 and a proximal end 104. A shaft assembly 106 extends between the distal end 102 and the proximal end 104.

The shaft assembly 106 may include a shaft 110 and a sheath 128. The shaft assembly 106 may include only the shaft 110 and the sheath 128. The shaft 110, in one example, may fill an interior of the sheath 128 such that no other shaft or component may be present or inserted into the sheath 128. The shaft assembly 106 may be configured to only include the shaft 110 and the sheath 128—other tubes or shafts are excluded from and not present in the shaft assembly 106 in one example. A body of the shaft 110 may be solid and not hollow.

The shaft 110 may be slidably disposed inside the sheath 128 such that it can be moved axially in distal and proximal directions. In one example, the sheath 18 may be an introducer sheath. The shaft 110 may be solid such that no other shaft may be disposed inside of the shaft 110.

A housing 118 is also disposed between the distal end 102 and the proximal end 104. Alternatively, the housing 118 may be located at or adjacent the proximal end 104. More generally, the housing 118 is typically located on the proximal side of the shaft assembly 106. The housing 118 may be integrated with or attached to the proximal end of the shaft assembly 106 or more specifically attached to a proximal end of the sheath 128.

The shaft 110 extends through the housing 118 and into the sheath 128 in one example such that the shaft 110 is slidably disposed in the shaft assembly 106 as previously stated. The shaft 110 can move distally and proximally relative to the housing 118, the sheath 128 and/or other aspects of the device 100.

A push button 112 (or other actuator such as a trigger) is disposed on a proximal end of the shaft 110. The push button 112 may an integral part of the shaft 112. The push button 112 is located outside of the housing 118 on a proximal side of the housing 118. The push button 112 can be actuated by a user pushing against the push button 112 to distally advance the shaft 110. A user can pull on the push button 112 to move the shaft 110 proximally. A trigger may alternatively be mounted on the housing 118 and configured to engage the shaft 110 to cause the shaft 110 to move distally relative to the sheath 128. The trigger may also be capable of moving the shaft 110 in a proximal direction.

The push button 112 may be configured such that the shaft 110 can be actuated by pressing on the push button 112 in a distal direction (e.g., towards the arteriotomy 158). Pressing on the push button 112 moves the shaft 110 in a distal direction. In one example, only the shaft 110 moves in a distal direction while the sheath 128 remains still. For example, a user may hold onto the ends of the sutures 150 and/or the sheath 128 while pressing on the shaft button 112 such that the shaft 110 is distally displaced.

The distal end 102 of the device 100 may be configured to trim the suture 150. More specifically, a distal end of the sheath 128 may be configured to cooperate with a distal end of the shaft 110 such that distal movement of the shaft 110 relative to the sheath 128 trims the ends of the suture 150 when the suture 150 are loaded in the device 100. In this manner, the device is configured to trim the suture 150.

The device 100 may also be configured to push the knot 160 to the arteriotomy 158. The distal end 102 of the device 100 includes a tip 116 having an opening 114 formed therein. The opening 114 extends from a distal end of the tip 116 to a proximal end of the tip 116. The opening 114 opens into an interior or lumen of the shaft assembly 106 and more specifically into an interior or lumen of the sheath 128. In one example, before the shaft 110 is actuated, there is a hollow or open space between the distal end of the shaft 110 and the tip 116.

When the suture 150 is loaded in the device 100, a user can grasp the device and, while holding one or more ends of the suture 150, push the knot 160 to the arteriotomy 158. The opening 114 is sized such that the suture 150 can pass therethrough. The opening 114, however, prevents the knot 160 from passing through the opening 114 and is thus configured to push the knot 160 towards the arteriotomy 158. The knot 160 may be slidable.

The shaft assembly 106 also includes a suture access port 108 formed in a portion of the side wall of the sheath 128. The suture access port 108 is typically formed near the distal end 102 of the device and may be adjacent the distal end of the sheath 128. The suture access port 108 may be located near or adjacent the tip 116. The suture access port 108 may be slightly spaced from a proximal end of the tip 116 or located a certain distance from the tip 116. The distance can be measured, by way of example, from either the distal or proximal end of the tip 116. In one example, the distance between a distal end of the suture access port 108 and the distal end of the tip 116 may determine where the sutures 150 are trimmed relative to the knot 160. Generally, the suture access port 108 is placed such that the suture 150, when trimmed, extends about a quarter-inch above the arteriotomy. The location of the suture access port 108, however, can be placed in other locations and the foregoing example is in no way limiting.

The opening 114 and the suture access port 108 form a suture path in the distal end 102 of the device 100. The suture path enables the ends of the suture 150 to be drawn into the interior of the shaft assembly 106 through the opening 114 and out of the interior of the shaft assembly 106 through the suture access port 108. The opening 114 is sized to allow the sutures 150 to be loaded in the device 100. At the same time, the opening 114 is sized to prevent the knot 160 from passing through the opening 114 as previously stated. This enables the distal end 102 and more specifically the tip 116 to push the knot 160 down to the arteriotomy 158.

The device 100 can be used, for example, to close the arteriotomy 158 (or other opening). In order to close the arteriotomy 158, the suture 150 is first placed in the vessel walls. The suture 150 or proximal ends of the suture 150 are then loaded into the device 100.

To load the suture 150, the device 100 includes a suture snare 120 configured to removably attach to the shaft assembly 106 or other portion of the device 100. The suture snare 120 may include a body 130. Grips 126 extending from the body 130 are configured to engage the shaft assembly 106. In one example, the grips 126 are flexible such that the suture snare 120 can be held in place on the shaft assembly 106 and be easily removed from the shaft assembly 106, for example by pulling. The grips 126 may be clips, or elastic grips.

The suture snare 120 also includes a snare 122 that ends in a loop 124. Ends of the sutures 150 are passed through the loop 124. The suture snare 120 is then detached from the shaft assembly 106 and the suture 150 held in or inserted through the loop 124 of the snare 122 is pulled into the opening 114 and out of the suture access port 108.

More specifically, the snare 122 is initially loaded in the suture path located in the distal end 102 of the device 100, for example by pushing the loop 124 into the interior of the sheath 128 via the suture access port 108 and then distally through the opening 114 until the loop 124 extends from the distal end of the device 100. When the suture 150 is held in or placed in the loop 124 and the snare 122 is withdrawn from the device 100, the loop 124 causes the ends of the suture 150 to then be pulled along the suture path and loaded in the suture path of the device 100. Loading the device 100 in this manner results in a situation where the ends of the suture 150 extend out of the suture access port 108 and can be manipulated (e.g., grasped, pulled, etc.) by a user.

After the suture 150 is loaded in the device 100, the tip 116 of the distal end 102 can push the knot 160 down to the arteriotomy 158. The ends of the suture 150 extending from the suture access port 108 can then be manipulated to tighten the knot 160. Once the knot 160 is appropriately fixed, the shaft 110 is then moved distally (e.g., by pressing on the push button 112) and the suture 150 is trimmed at the suture access port 108.

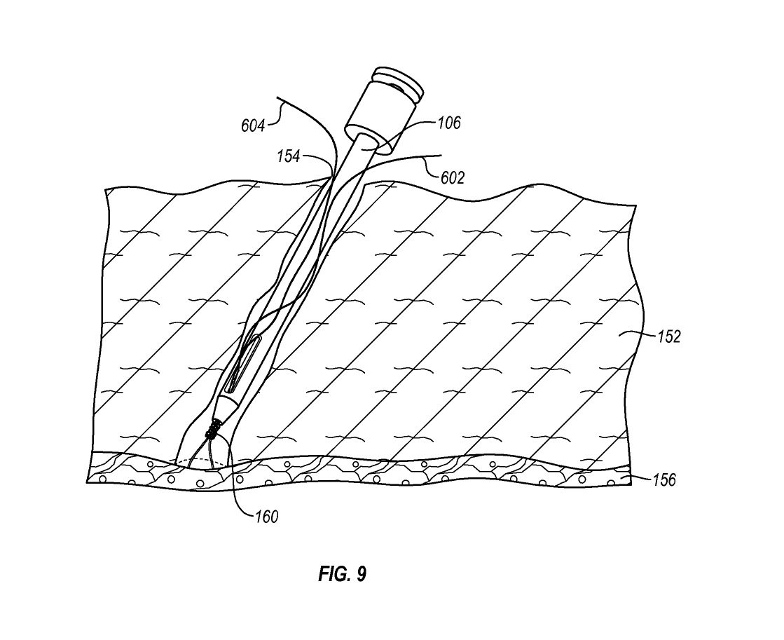

In one example, the distal end of the shaft 110 and the suture access port 108 cooperate to cut the suture 150, for example by a scissor action between the distal end of the shaft 110 and an edge of the suture access port 108. Actuating the shaft 110 can trim the sutures 150 below the puncture site 154. Once the suture 150 is trimmed, the device 110 can be withdrawn from the puncture site 154. Advantageously, the knot 160 can be secured and trimmed without over-dilating the puncture site 154. In one example, because the shaft assembly 106 only includes the sheath 128 and the shaft 110, the cross-sectional dimensions of the shaft assembly 106 can be smaller than conventional devices. In one example, a diameter of the shaft assembly 106 may be 0.082 inch or on the order of 2 millimeters. The diameter can be in a range, in one example from 1 mm to 3 mm. The dimensions could also be outside this range. Securing and tightening the knot are described in more detail with reference to FIG. 8.

FIG. 2 illustrates a cross sectional view of the device 100 and illustrates the shaft assembly 106. In FIG. 2, a snare 122 is loaded along a suture path 224 of the device 100. FIG. 2 illustrates that the shaft assembly 106 includes the shaft 110 that is slidably disposed inside the sheath 128. The shaft 110 has a distal end 206 and a proximal end 208. The proximal end 208 may attach to or be integral with the push button 112. Alternatively, the shaft 110 may have notches configured to engage with a trigger mechanism such that actuating the trigger advances the shaft 110 distally.

In one example, the shaft 110 is biased in a proximal direction by a spring 202 disposed inside the housing 118. The spring 202 (or other bias mechanism) has a distal end that pushes against a distal end of the housing 118. A proximal end 214 of the spring 202 pushes against the shaft 110 in a distal direction, for example against an extension 204 of the shaft 110. The extension 204 extends radially in one example from the shaft 110 and has sufficient area to engage the spring 202.

Because the distal end 206 of the shaft 110 cooperates with the suture access port 108 to trim the suture 150, the spring 202 biases the shaft 110 such that the shaft 110 does not move distally inadvertently and trim the suture 150 prematurely. The force of the spring 202, however, can be overcome by a user pressing on the push button 112. In another example, the housing 118 can be omitted and friction between the shaft 110 and the sheath 118 may be sufficient to prevent the suture 150 from being trimmed prematurely. In another example, the shaft 110 can simply be introduced into the sheath 128 when needed to trim the suture 150.

A fit between the shaft 110 and the sheath 128 may be configured such that the shaft 110 is in contact with the sheath 128. If necessary, the shaft 110 may be lubricated with an appropriate lubricant to facilitate movement of the shaft 110 relative to the sheath 128. Both the shaft 110 and the sheath 128 may be flexible as well and need not be made of metal. The shaft 110 may be solid or not hollow in one embodiment. The shaft 110 and the sheath 128 can both be entirely formed of an appropriate plastic or other material such as by way of example and not limitation stainless steel or PEEK (polyether ether ketone). The device 110 may use an introducer sheath as the sheath 128. As a result, the sheath 128 can be quite thin relative to conventional knot pushers/suture trimmers. In one example, sheath 128 may have a thickness between 1 mm to 3 mm, for example 0.82 inch. A diameter of the shaft 110 may be 0.038 inch or between 0.5 and 2 mm in some examples by way of example and not limitation.

The distal end 102 of the device 100 includes the tip 116, which includes the opening 114. While the lumen or interior 216 of the sheath 128 may be hollow or empty in order to accommodate distal movement of the shaft 110, the tip 116 may be more solid and only have the opening 114. In one example, a solid tip 116 (except for the opening 114 extending therethrough) can provide stability to the sheath 128 when trimming the sutures 150. In addition, the tip 116 is the portion of the device 100 that contacts the knot 160 and pushes against the knot 160.

In one example, the tip 116 may have a length 210 in a longitudinal direction of the shaft assembly 106. While the distal end 220 of the tip 116 pushes against the knot 160, a proximal end 218 of the tip 116 may act as a stop for distal movement of the shaft 110. A space 222 between the proximal end 218 of the tip and a distal end 232 of the suture access port 108 may also be provided. A length 210 of the tip 116 and the space 222 may be configured such that the sutures are trimmed, by way of example and not limitation, about one-fourth inch from the arteriotomy.

In one example, the sheath 128 has a distal end 230 and a proximal end 236. The suture access port 108, which has a proximal end 234 and a distal end 232, is formed in a portion of a side wall of the sheath 128 at a location adjacent the distal end 230 of the sheath 128. The distal end 230 is joined with or may be integrally formed with the tip 116.

The shaft 110 also has a distal end 206 and a proximal end 208. An outer diameter (or perimeter when non-circular) of the shaft 110 may be selected to permit the shaft to fit within the lumen 216 or interior of the sheath 128. The shaft 110 may have a close tolerance relationship with the sheath 128 while allowing the shaft 110 to move axially relative to the sheath 128. Axial movement of the shaft 110 may occur from a first position to a second position. The distal end 206 of the shaft 110 may be initially positioned at a first position that is located proximally of the distal end 232 of the suture access port 108. For example, the distal end 206 of the shaft 110 may be positioned between the distal end 232 and the proximal end 234 of the suture access port 108. The distal end 206 may be positioned proximally of the proximal end 234 of the suture access port 108. Actuation of the shaft 110 can trim the sutures loaded therein. The distal end 102 of the shaft assembly is configured to push the knot and the distal end 232 of the suture access port 108 can be configured to trim the suture 150 as the shaft 110 is moved from the first position to the second position, which may be located distally of the distal end 232 of the suture access port 108. For example, somewhere in the space 222.

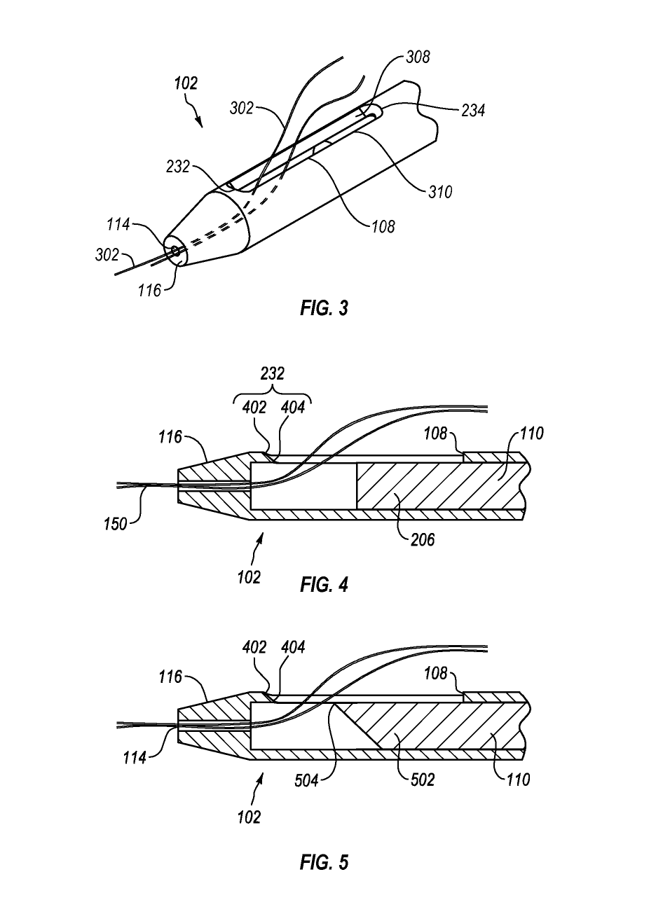

FIG. 3 illustrates a perspective view of the distal end 102 of the device 100. FIG. 3 illustrates a suture path 302 (shown as a dashed line) of the device 110. The suture path 302 is through the opening 114 into the interior of the device 110 and then through the suture access port 108.

FIG. 3 further illustrates that the suture access port 108 includes sides 308 and 310. The sides 308 and 310 run parallel to the longitudinal axis of the device 100 in one example. The suture access port 108 also includes a distal end 232, which may also be referred to as a knife end 232 and a proximal end 234. When pushing the knot 160 with the distal end of the tip 116, the knife end 232 may not contact the sutures extending from the suture access port 108 or may not contact the sutures in a manner sufficient to prematurely trim the sutures.

In one example, any of the ends 232, 234 or the sides 308, 310 may be configured as a knife end. For example, the shaft 110 may be placed such that the distal end of the shaft 110 is in contact with the tip 116. In this example, the shaft 110 may include a cavity to form part of the suture path 302. Proximal movement of the shaft 110 may then trim the suture 150 using the proximal end 234 as the knife end. Alternatively, rotation of the shaft 110 relative to the sheath 128 may cause one of the sides 308 or 310 to act as a knife edge.

FIG. 4 illustrates a cross sectional view of the distal end of the device 100 shown in FIG. 3 and further illustrates an example configuration of the knife end 232. FIG. 4 illustrates that the knife end 232 may include an edge 404 and a slanted surface 402 than runs from an exterior surface of the sheath 128 to the edge 404. The knife end 232 is configured to cooperate with the distal end 206 of the shaft 110 in a scissor action. When the shaft 110 is moved distally, the suture 150 is trimmed by a scissor action of the distal end 206 of the shaft 110 and the knife end 232 and more particularly the edge 404.

In one example, the sheath 128 may be sufficiently thin and a slant surface is not required. In this example, the thickness of the sheath 128 may be sufficient to act as a knife end 232.

The knife end 232 may be formed of a material that is more stiff than the rest of the sheath 128. Alternatively, the distal end area (extending towards the proximal end) of the sheath 128 may be formed of a material that is more stiff than the rest of the sheath 128. For example, the edge 404 may be a harder or stiff insert that is placed on the distal end of the suture access port 108. The insert may be metal as well.

The distal end 206 of the shaft 110 may be sized to engage with the knife end 232 to form an adequate scissor action. Forming the knife end or the distal end 232 of the suture access port 108 of a stiffer or harder plastic or of a metal may improve the trimming capability of the device 100. In one example, the edge 404 may be formed of metal that is deposited or attached to the knife end 232. A corresponding knife portion of the distal end of the shaft 110 may also be formed of metal in a similar manner.

FIG. 5 illustrates another cross sectional view of the distal end of the device 100. In FIG. 5, the distal end 502 of the shaft 110 includes a more pointed or sharper edge 504 on the distal end 502 of the shaft 110. The edge 504 cooperates with the edge 404 to trim the suture loaded in the device 100.

The device 100 is thus configured to push a knot and trim the suture. In one example, the orientation of the shaft 110 to the sheath 128 may be maintained. In one example, the shaft 110 may include a notch that engages with a corresponding protrusion (for example formed in the housing 118 to keep the shaft 110 oriented relative to the sheath 128 or more specifically to the suture access port 108. For example, the proximal end of the shaft 110 may include a notch that engages with a protrusion in the housing 118 or vice versa. Alternatively, the extenders 204 (which may be an integral part of the shaft 110) and spring 202 may be configured to keep the shaft 110 in a particular orientation relative to the suture access port 108. The extenders 204 are examples of an orientation mechanism configured to keep the shaft oriented with respect to the suture access port or other aspects of the device 100.

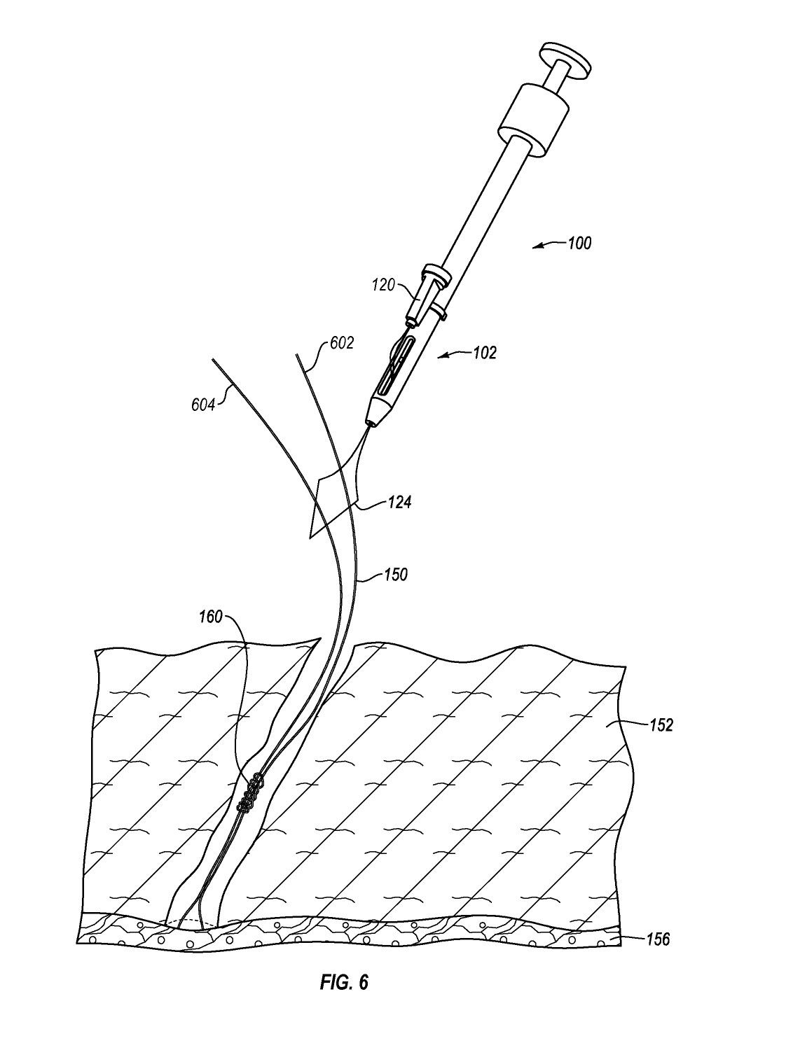

FIGS. 6-10 illustrate a method for closing an arteriotomy. FIG. 6 illustrates the device when ends 602 and 604 of the suture 150 are passed through the loop 124 of the suture snare 120. In this example, the suture 150 has been placed in the vessel walls surrounding the arteriotomy and a knot 160 has been formed in the suture 150. One of the ends 602 and 604 is a rail end of the suture 150.

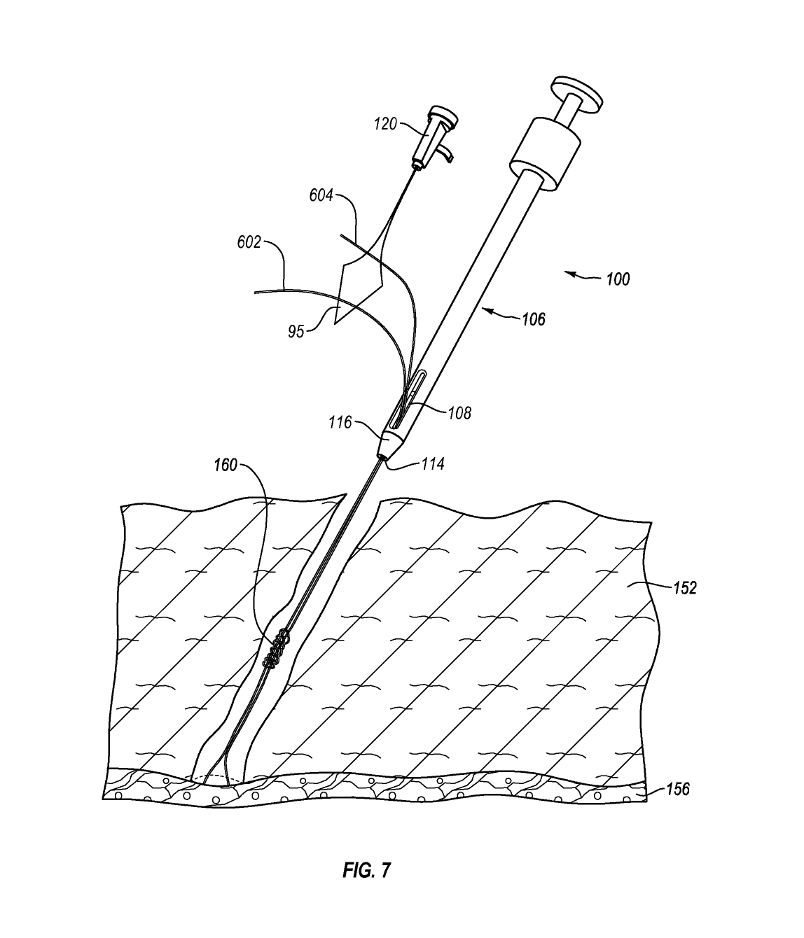

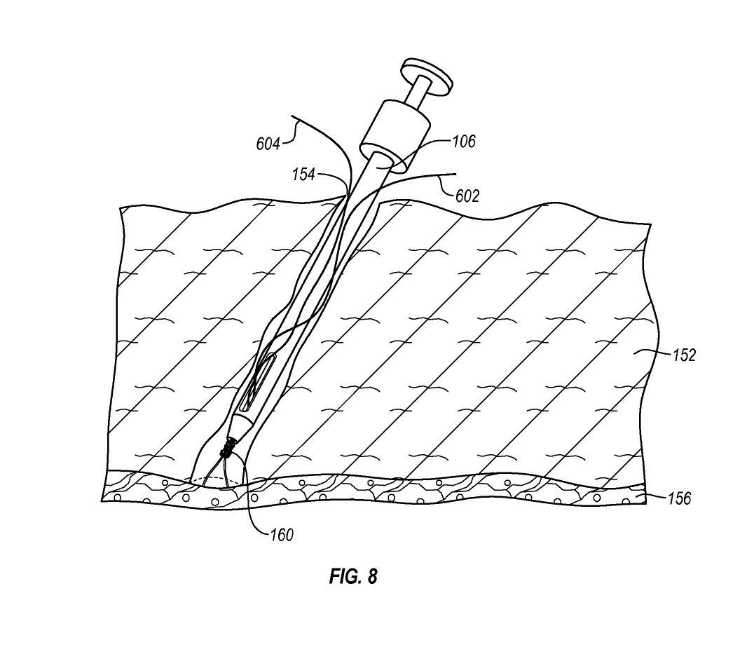

FIG. 7 illustrates the device 100 after the suture 150 has been loaded in the distal end of the device 100. In FIG. 7, the suture snare 120 has been removed from the device 100 and the ends 602 and 604 have been loaded in the device 100 by pulling the ends 602 and 604 held in the suture snare 120 through the opening 114 and out of the suture access port 108. The suture 150 is thus loaded in the suture path of the device 100.

FIG. 8 illustrates the knot being pushed down to the arteriotomy. In this example, a rail end 602 of the suture 150 may be held by the user while the device 100 is pushed distally. Pushing the device distally causes the tip 116 to engage the knot 160 and push the knot down to the arteriotomy in the vessel 156. The non-rail suture 604 can then by pulled (while securely holding the rail end 602 and the device 100 in place) to lock and tighten the knot 160 in place. When pushing the knot 160, the entire device (including the shaft assembly) is moved distally in one example. Relative movement of the shaft 110 to the sheath 128 is avoided to prevent trimming the suture 150 prematurely. In addition, the shaft 110 may not be introduced into the lumen of the sheath 128 until the knot 160 is ready to be trimmed.

FIG. 9 illustrates trimming the suture 150. In this example, the push button 112, and thus the shaft 110, is pressed in a distal direction. The sheath 128 may be held by a user in this example to advance the shaft 110 distally relative to the sheath 128. Alternatively, since the tip is pressed against the knot, the knot may provide sufficient resistance to simply advance the shaft 110 relative to the sheath 128. Actuating the push button 112 causes the shaft 110 to move distally. As previously described, a distal end of the shaft engages with the knife end or knife edge (e.g., the distal end) of the suture access port 108 to trim the suture 150.

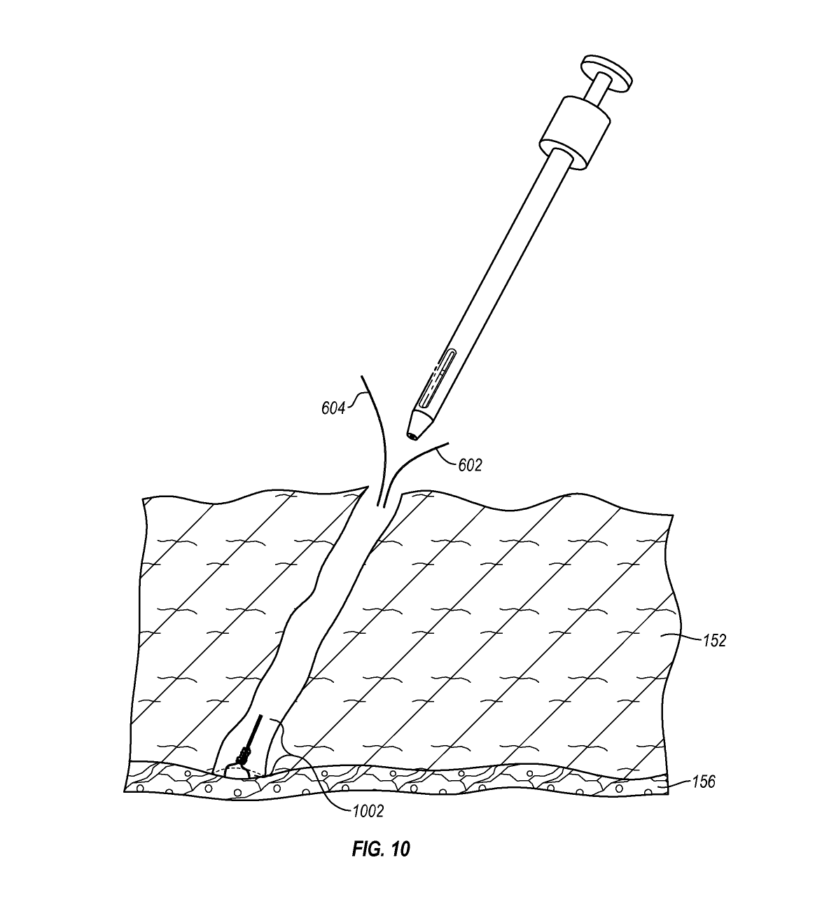

FIG. 10 illustrates removing the device 100 after the suture 150 is trimmed. After the suture is trimmed, the device 100 is withdrawn, leaving a knot in the suture 150 that closes the arteriotomy in the vessel 156. The knot is trimmed below a surface of the tissue 152 and may have a height 1002 of, by way of example only and not limitation, one-quarter inch. The height at which the suture 150 is trimmed can vary. The ends 604 and 602 can be discarded after trimming.

Advantageously, the knot can be pushed and secured without over-dilating the puncture site used to perform the preceding procedure. Because the device 100 has relatively few parts, the overall size can be reduce, which aids in preventing the puncture site from being over-dilated. In addition, the suture can be trimmed below the surface of the puncture site, allowing the puncture site to heal while the suture is slowly absorbed.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A device for delivering, securing and trimming a suture, the device comprising:
a shaft assembly extending between a distal end and a proximal end, the shaft assembly including:
a sheath having a proximal end, a distal end and a lumen having an inner diameter, the sheath also having a suture access port formed in a portion of a side wall of the sheath adjacent the distal end of the sheath, the suture access port having a proximal end and a distal end;
a shaft slidably disposed inside the sheath, the shaft having a proximal end, a distal end, and an outer diameter that is selected to permit the shaft to fit within the lumen of the sheath in a close tolerance relationship while allowing the shaft to move axially relative to the sheath, and the shaft being movable relative to the sheath between a first position, wherein the distal end of the shaft is positioned between the distal end and proximal end of the suture access port, and a second position, wherein the distal end of the shaft is positioned beyond of the distal end of the suture access port;
wherein the distal end of the shaft assembly is configured to push a knot formed in the suture; and
wherein the distal end of the shaft and the distal end of the suture access port are configured to trim the suture as the shaft is moved from the first position to the second position.

2. The device of claim 1, wherein the sheath comprises an introducer sheath.

3. The device of claim 1, wherein the shaft has a solid body.

4. The device of claim 1, the suture access port having a knife end, wherein a distal end of the shaft cooperates with the knife end to trim the suture.

5. The device of claim 4, wherein the knife end includes a first metal insert formed thereon and/or at least a portion of the distal end of the shaft includes a second metal insert, wherein the first metal insert and/or the second metal insert cooperate to trim the suture.

6. The device of claim 4, wherein the knife end is formed of a harder material than other portions of the sheath.

7. The device of claim 1, further comprising a tip disposed at a distal end of the device, the tip including an opening configured to allow the suture to pass through the opening while preventing a knot in the suture from passing through the opening.

8. The device of claim 1, wherein a proximal end of the shaft includes a push button configured to be pushed distally to advance the shaft distally inside the sheath.

9. The device of claim 1, further comprising a housing holding a biasing mechanism, wherein the biasing mechanism is configured to prevent premature distal movement of the shaft.

10. The device of claim 7, wherein the biasing mechanism includes a spring that engages with extenders formed on the shaft to bias the shaft in a proximal direction.

11. The device of claim 1, wherein the shaft and the sheath are both flexible.

12. A device for delivering and securing a suture, the device comprising:
a distal end having a tip configured to push a knot in the suture;
a proximal end;
a shaft assembly extending between the distal end and the proximal end, the shaft assembly including:
a sheath, the sheath having a suture access port; and
a shaft slidably disposed inside the sheath, wherein a distal end of the shaft cooperates with the suture access port to trim the suture after the knot is tightened.

13. The device of claim 12, wherein the shaft is a solid body and only the shaft is disposed inside the sheath, wherein the shaft substantially fills a lumen of the sheath.

14. The device of claim 12, wherein the tip and the suture access port form a suture path for the suture through the device, the tip having an opening sized to accommodate the suture and to prevent a knot in the suture from passing through the opening.

15. The device of claim 12, wherein the suture access port includes a knife edge, the knife edge performing a scissor action with the distal end of the shaft when the shaft is moved distally towards the tip.

16. The device of claim 15, wherein the knife edge is formed on a side of the suture access port, wherein the knife edge comprises a material that is harder than a material of the sheath.

17. The device of claim 16, wherein the distal end of the shaft is angled to form an edge that cooperates with the knife edge to trim the suture.

18. The device of claim 12, further comprising a housing holding a biasing mechanism, wherein the biasing mechanism is configured to prevent premature distal movement of the shaft.

19. The device of claim 18, wherein the biasing mechanism includes a spring that engages with extenders formed on the shaft to bias the shaft in a proximal direction.

20. The device of claim 12, wherein the shaft and the sheath are both flexible.

21. The device of claim 12, further comprising an orientation mechanism configured to keep the shaft oriented with respect to the suture access port.

22. The device of claim 12, wherein the suture access port is placed such that the suture can be trimmed below a puncture site.

23. A device for delivering and securing a suture, the device comprising:
a distal end having a tip configured to push a knot in the suture;
a proximal end having a housing, wherein a bias mechanism is located within the housing;
a shaft assembly extending between the distal end and the proximal end, the shaft assembly including:
a sheath, the sheath having a suture access port, wherein the tip and the suture cooperate to form a suture path through the device; and
a shaft slidably disposed inside the sheath, wherein only the shaft is slidably disposed inside the sheath
wherein a distal end of the shaft is advanced distally to cooperate with the suture access port to trim the suture after the knot is tightened,
the bias mechanism prevents the shaft from being prematurely advanced distally, and
the shaft passes through the housing such that a proximal end of the shaft is configured such that a user can advance the shaft distally.

24. The device of claim 23, wherein the suture access port includes a knife edge such that distal movement of the shaft trims the sutures by a scissor action between the knife edge and the distal end of the shaft.

25. The device of claim 23, wherein the shaft and the sheath are both flexible.

26. The device of claim 23, further comprising a snare removably mounted on the shaft assembly, the snare configured to draw the suture into the device such that the suture is loaded on the suture path.
